(19) United States
(12) Patent Application Publication
(10) Pub. No.: US 2024/0382141 A1
(43) Pub. Date: Nov. 21, 2024

(54) SYSTEMS AND METHODS FOR EXECUTIVE FUNCTION REHABILITATION

(71) Applicants: Arizona Board of Regents on Behalf of Arizona State University, Tempe, AZ (US); Mayo Foundation for Medical Education and Research, Rochester, MN (US)

(72) Inventors: Akuadasuo Ezenyilimba, Gilbert, AZ (US); Jennifer Wethe, Scottsdale, AZ (US); Nancy Cooke, Mesa, AZ (US); Troy McDaniel, Gilbert, AZ (US)

(21) Appl. No.: 18/668,093

(22) Filed: May 17, 2024

Related U.S. Application Data
(60) Provisional application No. 63/503,436, filed on May 19, 2023.

(51) Int. Cl. A61B 5/00; G16H 10/20; G16H 10/60; G16H 20/00; G16H 40/20

(52) U.S. Cl. CPC A61B 5/4088; G16H 10/20; G16H 10/60; G16H 20/00; G16H 40/20; A61B 5/742

(57) ABSTRACT

Various embodiments of a computer-implemented executive function rehabilitation application are disclosed.

### CROSS REFERENCE TO RELATED APPLICATIONS

This is a non-provisional application that claims benefit to U.S. Provisional Application Ser. No. 63/503,436, filed on May 19, 2023, which is herein incorporated by reference in its entirety.

### GOVERNMENT LICENSE RIGHTS

This invention was made with government support under Grant No. 1828010 awarded by the National Science Foundation. The government has certain rights in the invention.

### FIELD

The present disclosure generally relates to technologies associated with rehabilitation, and in particular, to systems and methods related to executive function rehabilitation.

### BACKGROUND

Currently individuals with a history of mild traumatic brain injury are less likely to seek treatment, do not know about available treatment, cannot afford treatment due to insurance or lack of insurance, and/or are not informed about available treatment options (Brown, Ackley, and Knollman-Porter, 2021). The common misconception that individuals with a history of mild traumatic brain injuries will automatically have ‘spontaneous recovery,’ is not a reality for many. For many mild traumatic brain injury individuals issues with post concussive symptoms can persist for months if not years after injury (Hadanny and Efrati, 2016), especially if there is no next level intervention to address rehabilitation needs. For many these post concussive symptoms can be referred to as post concussive syndrome or persistent post concussive syndrome, resulting in prolonged issues with an inability to plan effectively, multi-task, and impeded on overall quality of life.

Non-standardization of care and readily available knowledge has left a demographic of individuals to deal with the ramifications of ‘an invisible injury.’ With any other part of the body that you can physically see, such as an arm or a leg, when you injure, break, or fracture it, it is a no brainer that some form of rehabilitation is needed. But that is not always the case for mild traumatic brain injury. Many mild traumatic brain injury individuals may only be afforded first line care (i.e., emergency room), and from there are instructed to solely rest, prescribed medication, or may be misdiagnosed. The constant disconnect between first line care and next line care options has left this community with the inability to have access to treatment options until after the fact or until too late.

It is with these observations in mind, among others, that various aspects of the present disclosure were conceived and developed.

### SUMMARY

The following presents a simplified summary of various aspects described herein. This summary is not an extensive overview and is not intended to identify key or critical elements or to delineate the scope of the claims. The following summary merely presents some concepts in a simplified form as an introductory prelude to the more detailed description provided below. Corresponding apparatus, methods/processes, systems, and computer-readable media are also within the scope of the disclosure.

A computer-implemented application described herein, dubbed “Uburu,” is an executive function rehabilitation application intended for individuals with a history of mild traumatic brain injury. The intent behind the Uburu application is to be a rehab alternative and additive for the mild traumatic brain injury population and be a mode of rehab that can be transferred and applied to activities of everyday living. Uburu specifically focuses on executive function in the form of cognitive flexibility, organization, and planning, to help mild traumatic brain injury individuals address areas of executive dysfunction.

Stated another way, Uburu is an executive function rehabilitation application intended for individuals with a history of mild traumatic brain injury. The intent behind the Uburu application is to be a rehab alternative and additive for the mild traumatic brain injury population and be a mode of rehab that can be transferred and applied to activities of everyday living. Uburu specifically focuses on executive function in the form of cognitive flexibility, organization, and planning, to help mild traumatic brain injury individuals address areas of executive dysfunction. Currently individuals with a history of mild traumatic brain injury are less likely to seek treatment, do not know about available treatment, cannot afford treatment options due to insurance or lack of insurance, and/or are not informed about available treatment options. With Uburu individuals can use the application as a rehab additive or a rehab alternative when finances, insurance, or time are a limitation. Uburu aims to give users control within their TBI rehabilitation and apply their progress to everyday activities.

### BRIEF DESCRIPTION OF THE DRAWINGS

FIG. 1 is a flowchart illustrating an exemplary sequence of events associated with the executive function rehabilitation (Uburu) application described herein.

FIG. 2 is an illustration of exemplary components of the Uburu application described herein.

FIG. 3 is a simplified illustration of an exemplary computing device associated with examples of the system and application described herein.

FIG. 4A is a graphical representation showing initial and final experimental executive function assessment score average by group.

FIG. 4B is a graphical representation showing final experimental executive function assessment score average by group.

FIG. 5 is a graphical representation showing cognitive flexibility and executive function means by group.

FIG. 6A is a graphical representation showing “this or that” weekly game levels by experimental group.

FIG. 6B is a graphical representation showing “train of thoughts” weekly game levels by experimental group.

FIG. 6C is a graphical representation showing “keeping track” weekly game levels by experimental group.

FIG. 6D is a graphical representation showing “rank order” weekly game levels by experimental group.

FIG. 6E is a graphical representation showing “beat the value” weekly game levels by experimental group.

Corresponding reference characters indicate corresponding elements among the view of the drawings. The headings used in the figures do not limit the scope of the claims.

### DETAILED DESCRIPTION

Various embodiments herein relate to an inventive concept associated with an executive function rehabilitation application, also referred to herein as “Uburu.” In general, Uburu is an executive function rehabilitation application for mild traumatic brain injuries (TBI). Currently individuals with a history of mild traumatic brain injury are less likely to seek treatment, do not know about available treatment, cannot afford treatment options due to insurance or lack of insurance, and/or are not informed about available treatment options. With Uburu individuals can use the application as a rehab additive or a rehab alternative when finances, insurance, or time are a limitation. Uburu aims to give users control within their TBI rehabilitation and apply their progress to everyday activities.

In some examples, Uburu is specifically designed for mild traumatic brain injury individuals. Uburu aims to help train executive function, it is applicable to activities of daily living, and encourages users to access relevant resources, all while being a traumatic brain injury rehabilitation alternative and additive. Uburu aims to be a traumatic brain injury rehabilitation solution for those who are limited by time, finances, or insurance. By specifically focusing on executive function and mild traumatic brain injury users, individuals will be able to utilize the skills learned within the Uburu application and directly apply them to activities of daily living. Currently it is believed that there is no rehabilitation application that specifically targets (1) mild traumatic brain injury and (2) executive function. Every year there are approximately 2.8 million traumatic brain injuries/concussion (Nelson et al., 2019), of those 75 to 90% are classified as mild (Hadanny and Efrati, 2016). Unlike other applications that only train planing or organization or cognitive flexibility, Uburu focuses on all three areas. Uburu is a tool that would be beneficial to many individuals, but even more specifically it is an application that is designed for the mild traumatic brain injury community, a community that is often is neglected, uniformed, and misinformed of available resources. Uburu's focus is to allow the user to be aware and in control of their journey, by actively informing users of progress, setting goals, and adapting to the needs of the user. In addition the application, Uburu aims to inform individuals of local resources to utilize along their rehabilitation journey.

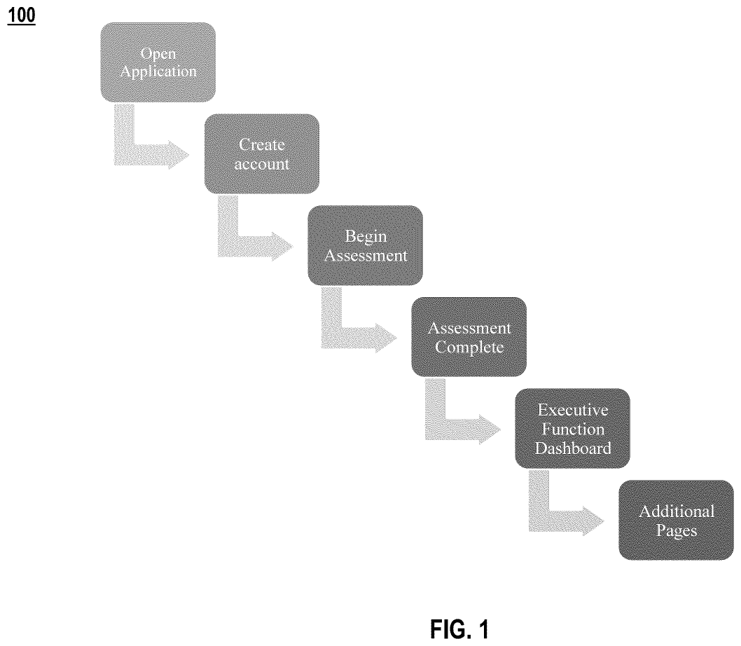

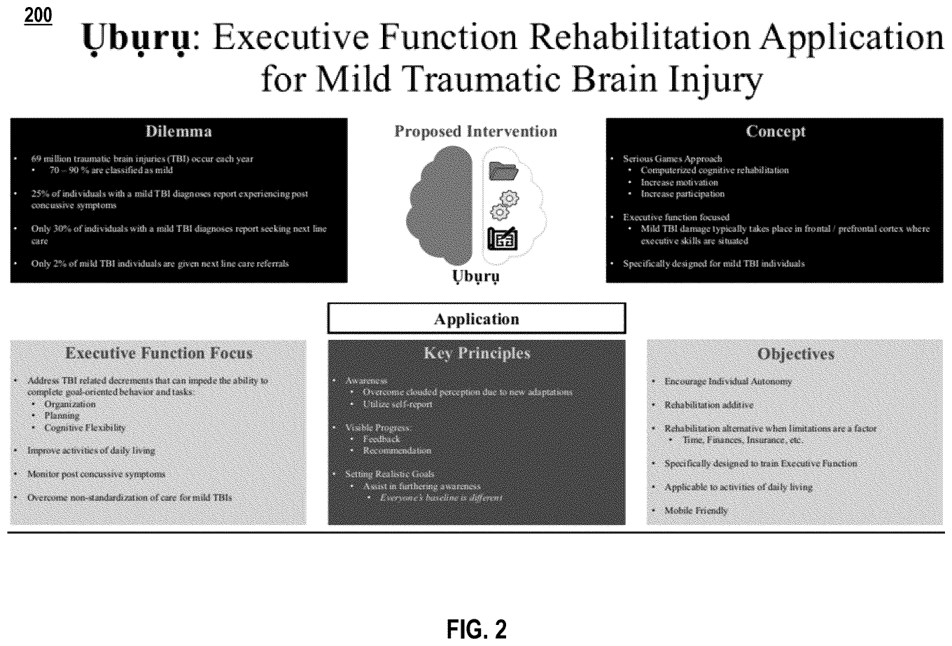

Referring to the exemplary process flow 100 and illustration 200 of FIGS. 1-2, Uburu is intended to be an application that allows users to be actively involved in their rehabilitation process. Upon accessing the Uburu application users will take the Uburu Assessment.

### Uburu Assessment

The initial Uburu Assessment will be utilized to give users a benchmark for a starting point in their journey. The Uburu Assessment consists of a traumatic brain injury/concussion history, self-report survey, planning assessment, organization assessment, and cognitive flexibility assessment.

The traumatic brain injury/concussion section of the assessment includes questions pertaining to user demographics (age and gender) and traumatic brain injury history (how long ago the injury occurred, age at injury, how the injury was acquired, etc.).

The self-report survey is broken down into two parts. The self-report survey utilizes a validity check by asking the same survey questions twice (once with negative language and once with positive language). The self-report survey focuses on questions about the users opinion in regard to how they view their ability to plan, organize, and switch between tasks.

From here participants begin the two-part planning section. The first part of the planning section gives users a real-life scenario in which they are instructed to read the information and utilize it to answer questions related to their ability to plan in terms of prioritizing. The second part of the planning section goes a step further and provides users with another real-life scenario in which they have to utilize available information to plan out a schedule, avoid potential conflicts, prioritize and meet requirement to achieve a higher-level goal.

The next part of the Uburu assessment is the organization section. This section is broken down into two parts as well. Users are given a list of items, from here they are instructed to order these items into a logical schedule that allows them to complete the overarching task. The second part of the organization section requires users to look at a disorganized list, in terms of order of events, decipher which items are urgent, and then answer questions about the items within the lists ability to be flexible and/or rescheduled.

The final part of the Uburu Assessment is the cognitive flexibility assessment. The cognitive flexibility section is broken down into three parts that has the user select the appropriate category for various words and objects. During the cognitive flexibility section of the assessment users are tasked with maintaining and switching between two tasks. As users progress through the cognitive flexibility section the workload increases.

### Weekly Tasks

At the conclusion of the Uburu Assessment, based on the users score Uburu will generate three focuses from the three executive function categories (cognitive flexibility, planning, and organization) into primary, secondary, and a tertiary focus. From there participants will now be able to access the Executive Function Dashboard.

The Executive Function Dashboard will be where users will be able to see their weekly games and survey. The Uburu application contains five possible games (This or That, Train of Thoughts, Beat the Value, Rank Order, and Keeping Track) and one weekly survey. Weekly tasks will be given to users in three sessions (also known as sets on the dashboard). Users will then be able to see their progress with their weekly games and weekly survey.

### This or That

The This or That game focuses on cognitive flexibility. The aim of the This or That game is task switching. Users will be instructed to select the most appropriate category for the word, or the object presented in the center of the screen. Based on the directions provided users will be selecting either a color or category for the presented item. At the end of each level users will see their score and time.

### Train of Thoughts

The Train of Thoughts game focuses on planning. Users will be given a story in which they will have to help decide what should happen first in order for the train to leave the station. Each cart attached to the train will represent a step/event.

Users will then list each step or event in the most logical order. At the end of each level users will see their time and the scenario that played out based on their decisions.

### Beat the Value

Beat the Value focuses on cognitive flexibility. Users will be presented with various equations (addition, subtraction, multiplication, and division). Based on the question asked users will decide between two equations which one is less than, greater than, or equal to the presented number, if none of the equations answer the presented question users will have the option to select none, which will be presented in the center of the two equations. At the end of each level users will see their score and time.

### Rank Order

The Rank Order game focuses on organization. Users will be responsible for organizing a list of items or events into the most logical schedule. As levels progress they will also have to decide which events are urgent, flexible or able to be rescheduled. At the end of each level users will see their score and time,

### Keeping Track

Keeping track incorporates cognitive flexibility, planning, and organization. Users will be presented an item in the center of the screen. Items presented to users will have slight differences. Based on these differences users will be responsible for selecting the most appropriate category. At the conclusion of each level users will then have to report how many items were in each category. At the end of each level users will see their score and time.

### Level Difficulty

Level progression will be based on the users scores from the previous week. If users are hitting the threshold score they will advance to the next level the next week. If users do not hit the threshold score users will repeat the previous weeks level, but a slightly different version or randomization depending on the game. At the conclusion of each level for each game, after seeing their score, users will set goals for their next session of each game. From here users will then see tips for their next session as well as recommendations in terms of resources. Recommendations will be provided to users who are not progressing or repeatedly not hitting threshold scores to encourage users to access available resources that could help within the rehabilitation journey.

### Resources

The resource page of the Uburu application will focus on local available resources in terms of support groups, insurance and other pertinent resources. Resources will also include evidence-based links for executive function such as cognitive flexibility, planning, and organization.

Some non-exemplary illustrative features include:
Uburu Assessment
Benchmark starting point—
TBI/concussion history: demographics and medical info about TBI. self-reporting survey: validity check, how user views their cognitive ability
planning assessment: users plan and prioritize tasks in two simulated real-life situations (ex: make a schedule, plan to reach a higher goal)
organizing assessment: order items in a logical way, reorganize disordered items
cognitive flexibility assessment: maintain or switch between tasks
Weekly tasks—
Benchmark assessment generates score and then users do weekly tasks to increase their score.
This or that game for cognitive flexibility
Train of thoughts game for planning
Beat the value game for cognitive flexibility
Rank order game for organization
Keeping track game for cognitive flexibility, planning, and organization
Difficulty levels increase as the user makes progress
App includes a resources page for support groups, insurance info, and other patient resources

### Task Level Descriptions

Exemplary task level descriptions are as follows:

### 1. Train of Thoughts

### Instructions: Read the story below

Level 1:
   5 carts
   Story:
      Set 1:
         Clayton has his second round interview for his dream job today at 9:15 AM in New York City. The train ride from Stamford to New York City takes 1 hour and 5 minutes.
         It is a 10 minute walk from the train station to the office in New York City, and in Stamford it takes Clayton 5 minutes to walk from his apartment building to the Stamford Station.
         It is currently 7:07 AM, Clayton is not dressed yet and has a history of low blood sugar when he doesn't eat. The next train leaves at 7:40 AM. Clayton also has to take his dog to his neighbor for the day.
         Click ‘Next’ to begin the tutorial.
      Set 2:
      Set 3:
Level 2:
   6 carts
   Story:
      Set 1:
      Set 2:
      Set 3:
Level 3:
   7 carts
   Story:
      Set 1:
      Set 2:
      Set 3:
Level 4:
   8 carts
   Story:
      Set 1:
      Set 2:
      Set 3:
Level 5:
   9 carts
   Story:
      Set 1:
      Set 2:
      Set 3:
      Level 6:
   10 carts
   Story:
      Set 1:
      Set 2:
      Set 3:
      Level 7:
   11 carts
   Story:
      Set 1:
      Set 2:
      Set 3:
Level 8:
   12 carts
   Story:
      Set 1:
      Set 2:
      Set 3:

### 2. This or That

Level 1:
   20 items
      Words
      Objects
   2 buttons
      Color
      Category
   Instructions: All three sets
      When the WORD appears CLICK THE COLOR of the word to the RIGHT; When the OBJECT appears CLICK THE CATEGORY it belongs to on the LEFT; Please click the NEXT button to BEGIN THE LEVEL
   3 colors
      Red
      Yellow
      Blue
   3 categories
      Food
      Sport
      Shape
   2 buttons
   Button specific location directions at the beginning
      Color to the right
      Category to the left
      Corrective advice
Level 2:
   30 items
      Words
      Objects
   2 buttons
      Color
      Category
   Instructions: All three sets
      When the WORD appears CLICK THE COLOR of the word to the RIGHT; When the OBJECT appears CLICK THE CATEGORY it belongs to on the LEFT; Please click the NEXT button to BEGIN THE LEVEL
   3 colors
      Red
      Yellow
      Blue
   3 categories
      Food
      Sport
      Shape
   Button specific location directions at the beginning
      Color to the right
      Category to the left
   Corrective advice
Level 3:
   30 items
      Words
      Objects
   2 buttons
      Color
      Category
   Instructions: All three sets
      When the WORD appears CLICK THE COLOR of the word; When the OBJECT appears CLICK THE CATEGORY; Please click the NEXT button to BEGIN THE LEVEL
   3 colors
      Red
      Yellow
      Blue
   3 categories
      Food
      Sport
      Shape
   No button specific location directions at the beginning
      Color to the left
      Category to the right
   No corrective advice
Level 4:
   2 rounds
      20 items each
         Set 1:
         Round one: Words only
         Round two: Words and Objects
         Set 2:
         Round one: Words and Objects
         Round two: Words only
         Set 3:
         Round one: Words
         Round two: Words and Objects
   Instructions:
      Set 1:
         Round one: When the WORD appears in BLACK CLICK THE CATEGORY; When the WORD appears in COLOR CLICK THE COLOR; Click NEXT to begin
         Round two: When the WORD appears CLICK THE COLOR of the word; When the OBJECT appears CLICK THE CATEGORY it belongs to; Please click the NEXT button to BEGIN THE LEVEL
      Set 2:
         Round one: When the WORD appears CLICK THE COLOR of the word; When the OBJECT appears CLICK THE CATEGORY it belongs to; Please click the NEXT button to BEGIN THE LEVEL
         Round two: When the WORD appears in BLACK CLICK THE CATEGORY; When the WORD appears in COLOR CLICK THE COLOR; Click NEXT to begin
      Set 3:
         Round one: When the WORD appears in BLACK CLICK THE CATEGORY; When the WORD appears in COLOR CLICK THE COLOR; Click NEXT to begin
         Round two: When the WORD appears CLICK THE COLOR of the word; When the OBJECT appears CLICK THE CATEGORY it belongs to; Please click the NEXT button to BEGIN THE LEVEL
   2 buttons
      Color
      Category
   4 colors
      Red
      Yellow
      Blue
      Black
   4 categories
      Food
      Sport
      Shape
      Transport
   No button specific location directions at the beginning
      Button type varies
   No corrective advice
Level 5:
   2 rounds
      20 items each
         Set 1:
         Round one: Objects only
         Round two: Words and Objects
         Set 2:
         Round one: Words and Objects
         Round two: Objects only
         Set 3:
         Round one: Words and Objects
         Round two: Objects only
   Instructions
      Set 1:
         Round one: When the OBJECT appears in BLACK CLICK THE CATEGORY; When the OBJECT appears in COLOR CLICK THE COLOR; Click NEXT to begin
         Round two: When the WORD appears CLICK THE COLOR of the word; When the OBJECT appears CLICK THE CATEGORY it belongs to; Please click the NEXT button to BEGIN THE LEVEL
      Set 2
         Round one: When the WORD appears CLICK THE COLOR of the word; When the OBJECT appears CLICK THE CATEGORY it belongs to; Please click the NEXT button to BEGIN THE LEVEL
         Round two: When the OBJECT appears in BLACK CLICK THE CATEGORY; When the OBJECT appears in COLOR CLICK THE COLOR; Click NEXT to begin
      Set 3:
         Round one: When the WORD appears CLICK THE COLOR of the word; When the OBJECT appears CLICK THE CATEGORY it belongs to; Please click the NEXT button to BEGIN THE LEVEL
         Round two: When the OBJECT appears in BLACK CLICK THE CATEGORY; When the OBJECT appears in COLOR CLICK THE COLOR; Click NEXT to begin
   2 buttons
      Color
      Category
   4 colors
      Red
      Yellow
      Blue
      Black
   4 categories
      Food
      Sport
      Shape
      Transport
   No button specific location directions at the beginning
      Button type varies
   No corrective advice
Level 6:
   3 rounds
      15 items each
         Set 1:
         Round one: Words only
         Round two: Object only
         Round three: Words and Objects
         Set 2:
         Round one: Objects only
         Round two: Words only
         Round three: Words and Objects
         Set 3:
         Round one: Words only
         Round two: Words and Objects
         Round three: Objects
   Instructions
      Set 1:
         Round one: When the WORD appears in BLACK CLICK THE CATEGORY; When the WORD appears in COLOR CLICK THE COLOR; Click NEXT to begin
         Round two: When the OBJECT appears in BLACK CLICK THE CATEGORY; When the OBJECT appears in COLOR CLICK THE COLOR; Click NEXT to begin
         Round three: When the WORD appears CLICK THE COLOR of the word; When the OBJECT appears CLICK THE CATEGORY it belongs; Please click the NEXT button to BEGIN THE LEVEL
      Set 2:
         Round one: When the OBJECT appears in BLACK CLICK THE CATEGORY; When the OBJECT appears in COLOR CLICK THE COLOR; Click NEXT to begin
         Round two: When the WORD appears in BLACK CLICK THE CATEGORY; When the WORD appears in COLOR CLICK THE COLOR; Click NEXT to begin
         Round three: When the WORD appears CLICK THE COLOR of the word; When the OBJECT appears CLICK THE CATEGORY it belongs; Please click the NEXT button to BEGIN THE LEVEL
      Set 3:
         Round one: When the WORD appears in BLACK CLICK THE CATEGORY; When the WORD appears in COLOR CLICK THE COLOR; Click NEXT to begin
         Round two: When the WORD appears CLICK THE COLOR of the word; When the OBJECT appears CLICK THE CATEGORY it belongs to; Please click the NEXT button to BEGIN THE LEVEL
         Round three: When the OBJECT appears in BLACK CLICK THE CATEGORY; When the OBJECT appears in COLOR CLICK THE COLOR; Click NEXT to begin
   2 buttons
      Color
      Category
   4 colors
      Red
      Yellow
      Blue
      Black
   4 categories
      Food
      Sport
      Shape
      Transport
   No button specific location directions at the beginning
      Button type varies
   No corrective advice
Level 7:
   3 rounds
      20 items each
         Set 1:
         Round one: Words only
         Round two: Object only
         Round three: Words and Objects
         Set 2:
         Round one: Words and Objects only
         Round two: Words only
         Round three: Objects only
         Set 3:
         Round one: Words only
         Round two: Objects only
         Round three: Words and Objects
   Instructions
      Set 1:
         Round one: When the WORD appears in BLACK CLICK THE CATEGORY; When the WORD appears in COLOR CLICK THE COLOR; Click NEXT to begin
         Round two: When the OBJECT appears in BLACK CLICK THE CATEGORY; When the OBJECT appears in COLOR CLICK THE COLOR; Click NEXT to begin
         Round three: When the WORD appears CLICK THE COLOR of the word; When the OBJECT appears CLICK THE CATEGORY it belongs to; Please click the NEXT button to BEGIN THE LEVEL
      Set 2:
         Round one: When the WORD appears CLICK THE COLOR of the word; When the OBJECT appears CLICK THE CATEGORY it belongs to; Please click the NEXT button to BEGIN THE LEVEL
         Round two: When the WORD appears in BLACK CLICK THE CATEGORY; When the WORD appears in COLOR CLICK THE COLOR; Click NEXT to begin
         Round three: When the OBJECT appears in BLACK CLICK THE CATEGORY; When the OBJECT appears in COLOR CLICK THE COLOR; Click NEXT to begin
      Set 3:
         Round one: When the WORD appears in BLACK CLICK THE CATEGORY; When the WORD appears in COLOR CLICK THE COLOR; Click NEXT to begin
         Round two: When the OBJECT appears in BLACK CLICK THE CATEGORY; When the OBJECT appears in COLOR CLICK THE COLOR; Click NEXT to begin
         Round three: When the WORD appears CLICK THE COLOR of the word; When the OBJECT appears CLICK THE CATEGORY it belongs to; Please click the NEXT button to BEGIN THE LEVEL
   4 buttons
      2 Color
      2 Category
   4 colors
      Red
      Yellow
      Blue
      Black
   4 categories
      Food
      Sport
      Shape
      Transport
   No button specific location directions at the beginning
      Button type varies
   No corrective advice
Level 8:
   3 rounds
      20 items each
         Set 1:
         Round one: Words only
         Round two: Objects only
         Round three: Words and Objects
         Set 2:
         Round one: Words and Objects only
         Round two: Objects only
         Round three: Words only
         Set 3:
         Round one: Words only
         Round two: Words and Objects
         Round three: Objects only
   Instructions
      Set 1:
         Round one: When the WORD appears in BLACK CLICK THE CATEGORY; When the WORD appears in COLOR CLICK THE COLOR; Click NEXT to begin
         Round two: When the OBJECT appears in BLACK CLICK THE CATEGORY; When the OBJECT appears in COLOR CLICK THE COLOR; Click NEXT to begin
         Round three: When the WORD appears CLICK THE COLOR of the word; When the OBJECT appears CLICK THE CATEGORY it belongs to; Please click the NEXT button to BEGIN THE LEVEL
      Set 2:
         Round one: When the WORD appears CLICK THE COLOR of the word; When the OBJECT appears CLICK THE CATEGORY it belongs to; Please click the NEXT button to BEGIN THE LEVEL
         Round two: When the OBJECT appears in BLACK CLICK THE CATEGORY; When the OBJECT appears in COLOR CLICK THE COLOR; Click NEXT to begin
         Round three: When the WORD appears in BLACK CLICK THE CATEGORY; When the WORD appears in COLOR CLICK THE COLOR; Click NEXT to begin
      Set 3:
         Round one: When the WORD appears in BLACK CLICK THE CATEGORY; When the WORD appears in COLOR CLICK THE COLOR; Click NEXT to begin
         Round two: When the WORD appears CLICK THE COLOR of the word; When the OBJECT appears CLICK THE CATEGORY it belongs to; Please click the NEXT button to BEGIN THE LEVEL
         Round three: When the OBJECT appears in BLACK CLICK THE CATEGORY; When the OBJECT appears in COLOR CLICK THE COLOR; Click NEXT to begin
   6 buttons
      3 Color
      3 Category
   4 colors
      Red
      Yellow
      Blue
      Black
   4 categories
      Food
      Sport
      Shape
      Transport
   No button specific location directions at the beginning
      Button type varies
   No corrective advice

### 3. Beat the Value

Level 1:
   20 values
   Instructions: present for each value
      Set 1: Which equation is greater than the sum?
      Set 2: Which equation is less than the sum?
      Set 3: Which equation is equal to the sum?
   3 buttons
      2 equations
      1 for none
   Equation type
      Addition
   Corrective advice
Level 2:
   20 values
   Instructions: present for each value
      Set 1: Which equation is greater than the sum?
      Set 2: Which equation is less than the sum?
      Set 3: Which equation is equal to the sum?
   3 buttons
      2 equations
      1 for none
   Equation type
      Subtraction
   Corrective advice
Level 3:
   20 values
   Question only present at beginning of each set
   Instructions: displayed only at beginning of round
      Set 1: Which equation is greater than the sum?
      Set 2: Which equation is less than the sum?
      Set 3: Which equation is equal to the sum?
   3 buttons
      2 equations
      1 for none
   Equation type
      Multiplication
   No corrective advice
Level 4:
   20 values
   Question only present at beginning of each set
   Instructions: displayed only at beginning of round
      Set 1: Which equation is greater than the sum?
      Set 2: Which equation is less than the sum?
      Set 3: Which equation is equal to the sum?
   3 buttons
      2 equations
      1 for none
   Equation type
      Division.
   No corrective advice
Level 5:
   2 rounds
      15 values each
   Question only present at beginning of each round
   Instructions: displayed only at beginning of round
      Set 1.
         Round one: Which equation is greater than the sum?
         Round two: Which equation is less than the sum?
      Set 2.
         Round one: Which equation is less than the sum?
         Round two: Which equation is equal to the sum?
      Set 3
         Round one: Which equation is greater than the sum?
         Round two: Which equation is equal to the sum?
   3 buttons
      2 equations
      1 for none
   Equation type
      Round one: Addition
      Round two: Multiplication
   No corrective advice
Level 6:
   2 rounds.
      20 values each
   Question only present at beginning of each round
   Instructions: displayed only at beginning of round
      Set 1
         Round one: Which equation is equal to the sum?
         Round two: Which equation is greater than the sum?
      Set 2
         Round one: Which equation is less than the sum?
         Round two: Which equation is equal to the sum?
      Set 3
         Round one: Which equation is greater than the sum?
         Round two: Which equation is less than the sum?
   3 buttons
      2 equations
      1 for none
   Equation type
      Round one: Subtraction
      Round two: Division
   No corrective advice
Level 7:
   3 rounds
      15 values each
   Question only present at beginning of each round
   Instructions: displayed only at beginning of round
      Set 1
         Round one: Which equation is equal to the sum?
         Round two: Which equation is greater than the sum?
         Round three: Which equation is less than the sum?
      Set 2
         Round one: Which equation is less than the sum?
         Round two: Which equation is equal to the sum?
         Round three: Which equation is greater than the sum?
      Set 3
         Round one: Which equation is greater than the sum?
         Round two: Which equation is less than the sum?
         Round three: Which equation is equal to the sum?
   3 buttons
      2 equations
      1 for none
   Equation type
      Round one: Subtraction
      Round two: Addition
      Round three: Division
   No corrective advice
Level 8:
   4 rounds
      15 values each
   Question only present at beginning of each round
   Instructions: displayed only at beginning of round
      Set 1
         Round one: Which equation is greater than the sum?
         Round two: Which equation is less than the sum?
         Round three: Which equation is equal to the sum?
      Set 2
      Round one: Which equation is equal to the sum?
         Round two: Which equation is greater than the sum?
         Round three: Which equation is less than the sum?
      Set 3
         Round one: Which equation is less than the sum?
         Round two: Which equation is equal to the sum?
         Round three: Which equation is greater than the sum?
   3 buttons
      2 equations
      1 for none
   Equation type
      Round one: Addition
      Round two: Subtraction
      Round three: Multiplication
      Round four: Division
   No corrective advice

### 4. Rank Order

### Instructions: Organize the list below into a logical schedule

Level 1:
   5 items
Level 2:
   6 items
Level 3:
   2 rounds
      7 items
Level 4:
   2 rounds
      8 items
   1 Column
      Urgent/Important
Level 5:
   2 rounds
      9 items
   2 Columns
      Flexible
      Urgent/Important
Level 6:
   2 rounds
      10 items
   2 Columns
      Flexible
      Reschedule
Level 7:
   2 rounds
      11 items
   3 Columns
      Flexible
      Reschedule
      Urgent/Important
Level 8:
   3 rounds
      12 items
   3 Columns
      Flexible
      Reschedule
      Urgent/Important

### 5. Keeping Track

### Instructions: Which category does the item belong to?

Level 1:
   20 items
   2 categories
   Corrective advice
Level 2:
   25 items
   2 categories
   Corrective advice
Level 3:
   30 items
   2 categories
   No corrective advice
Level 4:
   2 rounds
      15 items each
   2 categories
   No corrective advice
Level 5:
   2 rounds
      20 items each
   2 categories
   No corrective advice
Level 6:
   2 rounds
      20 items each
   3 categories
   No corrective advice
Level 7:
   3 rounds
      15 items each
   3 categories
   No corrective advice
Level 8:
   3 rounds
      20 items each
   3 categories
   No corrective advice

### EXEMPLIFICATION

As depicted in the Examples below, in certain exemplary embodiments, a computer-implemented system for executive function rehabilitation is conducted according to a serious games approach. It will be appreciated that, although the general methods depict the computer-implemented system of certain approaches of the present invention, the following general methods, and other methods known to one of ordinary skill in the art, can be applied to all approaches for executive function rehabilitation, as described herein.

### Design

The executive function study was a 2×2 (Group-between×Test within) mixed experimental design. Both the experimental and control group took the initial and final assessments, which consisted of both a clinical (CNS Vital Signs) and experimental assessments. Only the experimental group partook in the weekly experimental intervention.

### Participants

To determine the necessary number of participants for this study, an a priori power analysis was conducted using G*Power3 to evaluate the difference between the means of 2-group by 2-test (pre and post-test) using an F-test, a medium effect size (θₚ²=0.06), and an alpha of 0.05. Based on the results, a total sample of 34participants with two equal-sized groups of n=17 is required to achieve a power of 0.80. At the conclusion of the study a total of 27 participants successfully completed the study with n=13 for the control group, and n=14 for the experimental group.

To be eligible for this study, participants had to be at least 18 years of age, 6-months post mTBI, with normal to corrective vision. All participants within this study experienced a mTBI one of four ways, motor vehicle accident, physical altercation, fall, or sports related. Participants were grouped into six age categories (18-25, 26-30, 31-40, 41-50, 51-60, and 61+).

The average age group of the experimental group was 31-40, and the average age group of the control group was 26-30. The study included a total of 14 females, and 13 males. Twenty-five participants reported experiencing a mTBI more than a year ago, and two participants reported experiencing a mTBI less than a year ago (see Table 1).

TABLE 1
Demographics of experimental and control group.

| Group | Experimental | Control |
|---|---|---|
| Average age group: | 31-40 | 26-30 |
| Gender: | 6 females | 8 females |
| | 8 males | 5 males |
| When TBI occurred: | 13; <a year ago | 12; <a year ago |
| | 1; >a year ago | 1; >a year ago |
| Average age at injury: | 26.57 years of age | 20.53 years of age |
| Average number of TBIs: | 3.28 TBIs/person | 1.77 TBIs/person |
| How TBI occurred: | 4 Motor vehicle | 3 Motor vehicle |
| | 1 Physical altercation | 1 Physical altercation |
| | 9 Sports related | 7 Sports related |
| | | 2 Fall |
| Medical Treatment: | Yes: 11 | Yes: 4 |
| | No: 3 | No: 9 |

Participants were each given a participant ID number that was used for all participant data. Participant data for the study was stored on a secure google drive, which was not associated with any identifiable information. Participants were also randomly assigned to the experimental and control groups. A random number generator was utilized to assist in the random assignment of participants.

A list of thirty-four slots was created and a random number between 1 and 50 was then associated with each slot. If the number slot was filled with the number 25 or less that slot was then assigned to the experimental group; if the number slot was filled with a number greater than twenty-five that slot was then assigned to the control group. Participants in both groups were all recruited via three methods using a recruitment flyer.

After the initial flyer was circulated participants completed a pre-screening study questionnaire. Upon completion of the pre-screening questionnaire eligible participants were contacted via e-mail and asked to select a time for a pre-study information session. During the information session participants were informed of which group they were randomly assigned to, study requirements, and completed their consent forms. At the conclusion of each informational session participants were sent their initial assessments. Participants were not made aware of the treatment conditions for the group they were not assigned to.

### Initial and Final Assessments

The initial and final assessments consisted of two parts: validated clinical assessment and experimental executive function assessment. The clinical assessment was delivered to participants from the database CNS (Computerized Neurocognitive Assessment) Vital Signs.

The experimental executive function assessment was delivered to participants via Talent LMS (Learning Management System) using iSpring Quizmaker SCORM (Shareable Content Object Reference Model). The initial experimental executive function assessment consisted of three parts: demographics, self-report survey, and executive function assessments. The experimental executive function assessment is intended to gauge an individuals' ability to engage in tasks that require the ability to plan, organize, and engage in cognitive flexibility. Participants overall scores for the experimental executive function assessment were calculated by averaging the participant's scores on the in the aforementioned three sections, planning, organization, and cognitive flexibility.

At the conclusion of the initial assessment, experimental group participants received three scores that were then ranked based on primary, secondary, and tertiary focuses related to planning, organizing, and cognitive flexibility, see Table 3. At the conclusion of the six-week study all participants in both the experimental and control group retook both assessments. For the purposes of this study, the experimental executive function assessment focuses (primary, secondary, and tertiary) were used for participant knowledge solely.

TABLE 2
Experimental Steps.

| Step | Component | Description |
|---|---|---|
| STEP 1: Week 1 | Complete Initial | Clinical |
| | Assessment | Assessment: CNS |
| | | Vital Signs |
| | | Executive Function |
| | | (Uburu) |
| | | Assessment |
| STEP 2: Week 1 | Experimental group | Focuses will be |
| | receives Executive | planning, |
| | Function Focus; primary, | organization, and |
| | secondary, and tertiary | cognitive flexibility |
| STEP 3: Weeks 2-5 | Participate in weekly tasks | 5 games × 3 sessions |
| *Experimental | | per week |
| group only* | | |
| STEP 4: Week 6 | Complete Final | Clinical |
| | Assessment | Assessment: CNS |
| | | Vital Signs |
| | | Executive Function |
| | | (Uburu) |
| | | Assessment |

### Sessions

For six-weeks participants within the experimental group had three sessions per week in which they participate in five different games (three cognitive flexibility, one planning, and one organization) during each session (e.g., beat the value, keeping track, this or that, train of thoughts, rank order). Participants were also reminded to complete these weekly sessions via email or text depending on the participants preference.

As participants progressed throughout the week, game level difficulty either increased, decreased, or remained the same depending on the participants scores from the previous week. At the conclusion of each week participants also completed a weekly self-report survey.

During weeks 2-5 all participants within the experimental group participated in three sessions per week. To start off the study all participants began at level one for all five executive function games, there were a total of eight potential levels for each game. In order for participants to advance to the next level participants had to receive a score of 80 percent or higher. With the beta stage of the application being an accelerated version of the application's intended purpose, participants were capable of playing all five games at various levels depending on their individual progression on each game.

During participant's first attempt at level one and two, if the participant did not receive a passing score of 80% they would continue to remain at the level they were at until they were capable of reaching a passing score, rather than being limited to the standard three attempt rule that was present in levels 3-8. During level's one and two participants also received corrective feedback, and participants were made aware of their running score.

After participants reached level 3 they were then only allowed three attempts at each level to pass before being moved back down a level. During levels 3-8 corrective feedback was removed, and participants did not see their scores until the end each game. At the end of each game levels 1-8, all participants were encouraged but not forced to set goals related to target time and score for their next session of each game.

If participants were capable of completing all eight levels of a game before the conclusion of the study they were then exempt from the game for the remainder of the week. At the beginning of each week participants were then reassigned the games the leveled out of, and if they received a passing score again of 80% or better they were then again exempt from that game for the remainder of the week.

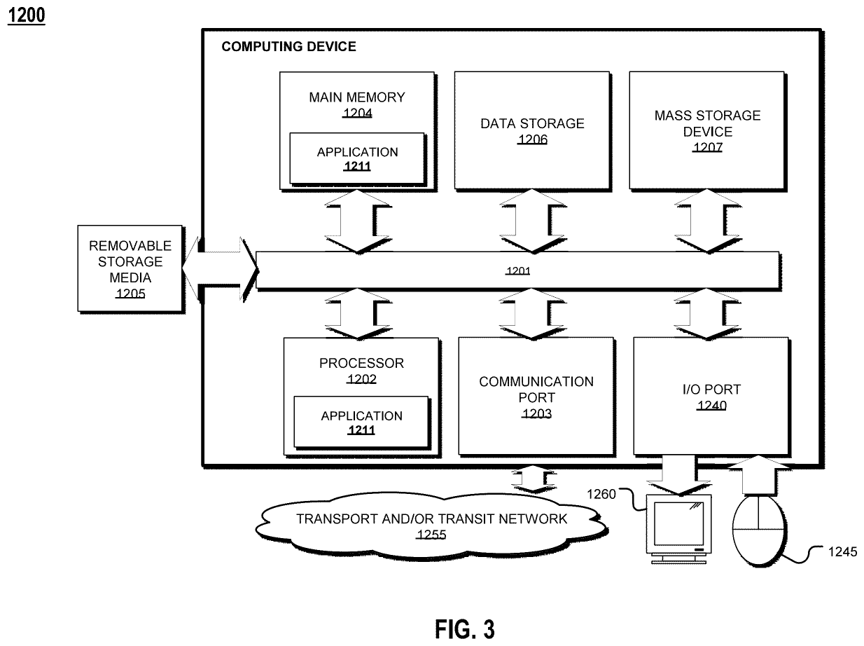

Referring to FIG. 3 a computing device 1200 is illustrated which may be configured, via one or more of an application 1211 (e.g., Uburu) or computer-executable instructions, to execute functionality described herein. More particularly, in some embodiments, aspects of the executive function rehabilitation application herein may be translated to software or machine-level code, which may be installed to and/or executed by the computing device 1200 such that the computing device 1200 is configured to execute functionality described herein. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

### Exemplary Computing Device

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media 1207 that includes removable/non-removable media and volatile/nonvolatile media and/or tangible media, but excludes transitory propagated signals. Computer-readable media 1207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 1204 includes computer storage media in the form of volatile/nonvolatile memory such as read only memory (ROM) and random access memory (RAM). A basic input/output system (BIOS), containing the basic routines that help to transfer information between elements within the computing device 1200 (e.g., during start-up) is typically stored in ROM. RAM typically contains data and/or program modules that are immediately accessible to and/or presently being operated on by processor 1202. Further, data storage 1206 in the form of Read-Only Memory (ROM) or otherwise may store an operating system, application programs, and other program modules and program data.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201 but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be implemented in a networked or cloud-computing environment using logical connections of a network interface 1203 to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computing device 1200 may be connected to a public and/or private network through the network interface 1203. In such embodiments, a modem or other means for establishing communications over the network is connected to the system bus 1201 via the network interface 1203 or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include

desktop computers, laptops, tablets e-readers, personal digital assistants, smartphones, gaming devices, servers, and the like. The computing devices may access computer-readable media that include computer-readable storage media and data transmission media. In some embodiments, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

### Results

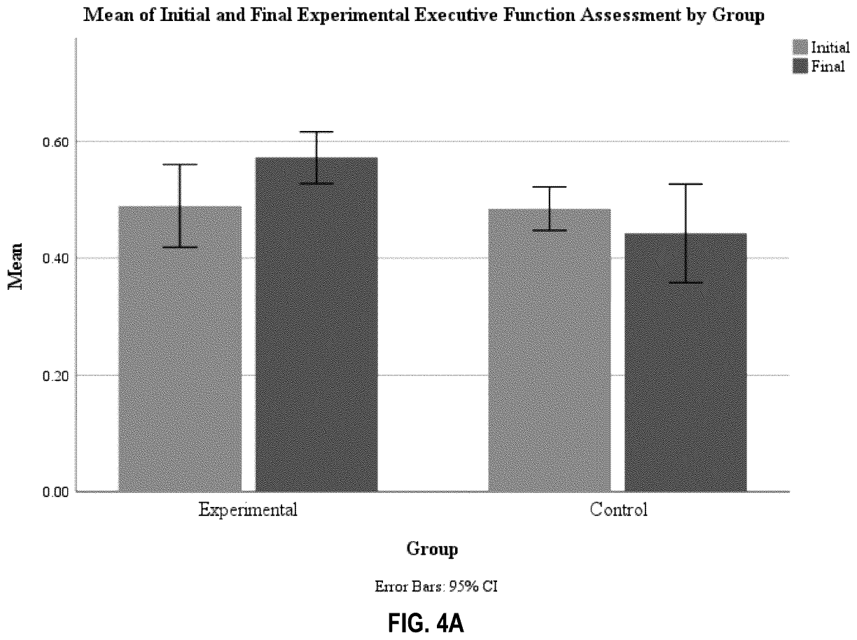

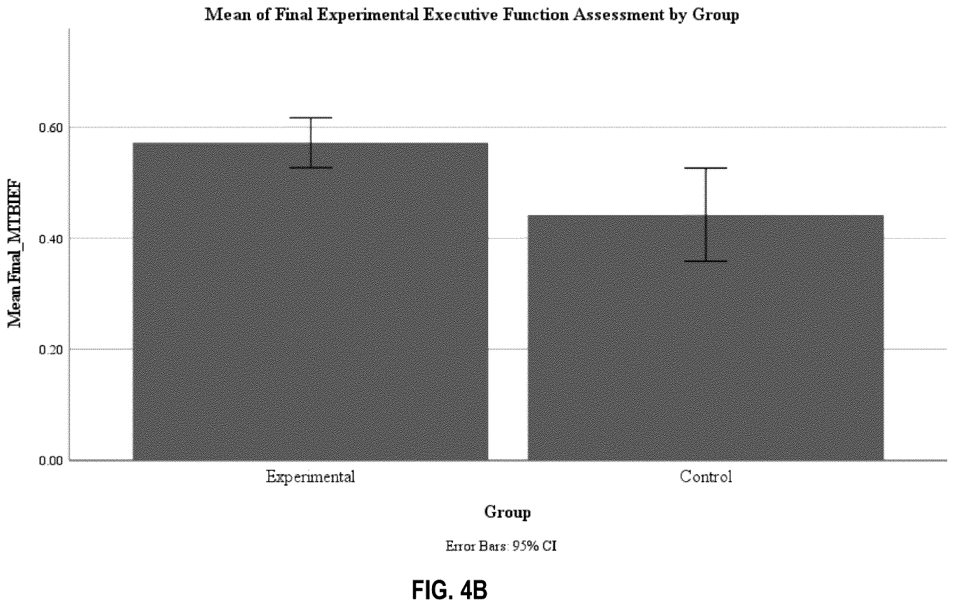

The experimental group's average score on the experimental executive function assessment was higher than the control group (see FIGS. 4A and 4B). To analyze the difference a one-way ANOVA was conducted to compare the means of the control and experimental groups final experimental executive function assessment scores. As a result, there was statistical significance between the groups resulting in, F(1,25)=9.109, p=0.006<0.05 (see Table 3).

TABLE 3
Experimental Executive Function
Assessment Scores One-Way ANOVA.

| Final_MTBIEF | Sum of Squares | df | Mean Square | F | Sig. |
|---|---|---|---|---|---|
| Between Groups | 0.114 | 1 | 0.114 | 9.109 | 0.006 |
| Within Groups | 0.312 | 25 | 0.012 | — | — |
| Total | 0.425 | 26 | — | — | — |

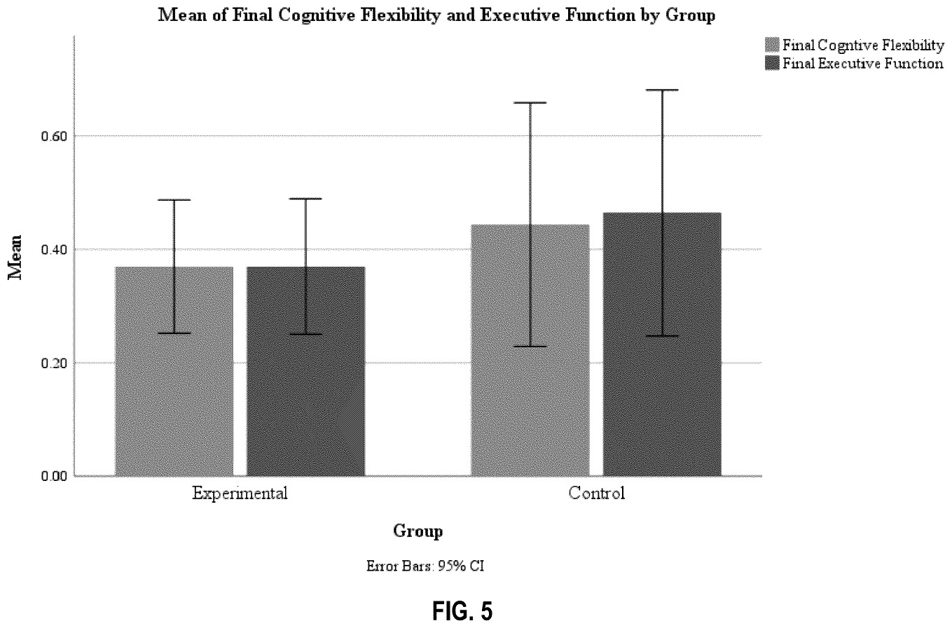

In analyzing the final clinical assessment scores between the experimental and control group, participants executive function and cognitive flexibility scores were assessed (see FIG. 5). To analyze if there was a difference between the group a one-way ANOVA was conducted to compare the means of the control and experimental groups final executive function and cognitive flexibility scores from the clinical assessment (see Table 4).

TABLE 4
Clinical Assessment: Cognitive Flexibility and
Executive Function Scores One-Way ANOVA.

| | | Sum of | | Mean | | |
|---|---|---|---|---|---|---|
| | | Squares | df | Square | F | Sig. |
| Final_CF | Between Groups | 0.036 | 1 | 0.036 | 0.475 | 0.497 |
| | Within Groups | 1.796 | 24 | 0.075 | — | — |
| | Total | 1.831 | 25 | — | — | — |
| Final_EF | Between Groups | 0.058 | 1 | 0.058 | 0.762 | 0.392 |
| | Within Groups | 1.838 | 24 | 0.077 | — | — |
| | Total | 1.896 | 25 | — | — | — |

The results yielded for the cognitive flexibility portion of the clinical assessment were F(1,24)=0.475, p=0.497>0.05. and the results for the executive function portion of the clinical assessment were F(1,24)=0.762, p=0.392>0.05. Unlike the experimental executive function assessment there was no statistical significance for the clinical assessment when it came to executive function and cognitive flexibility scores.

### This or That

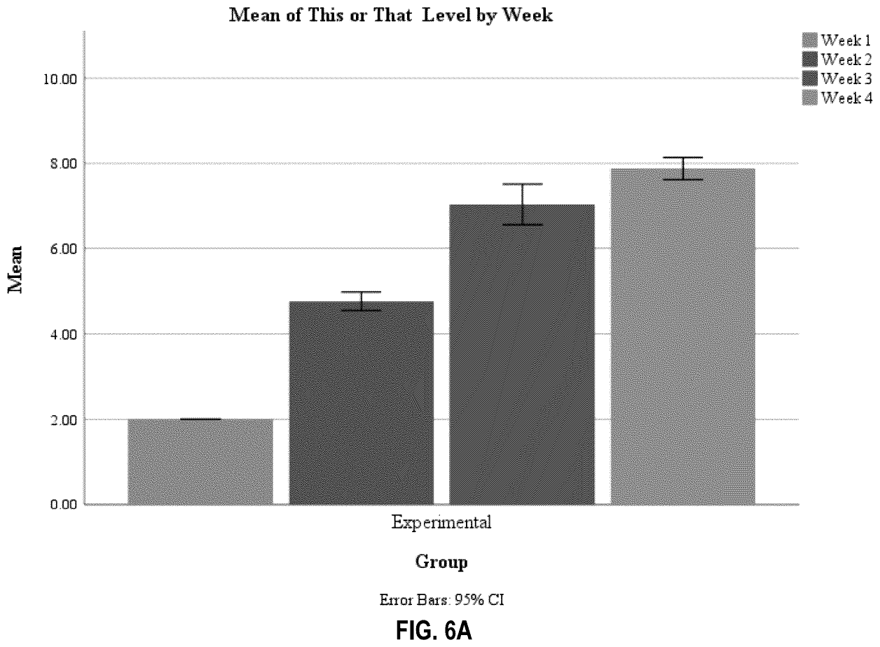

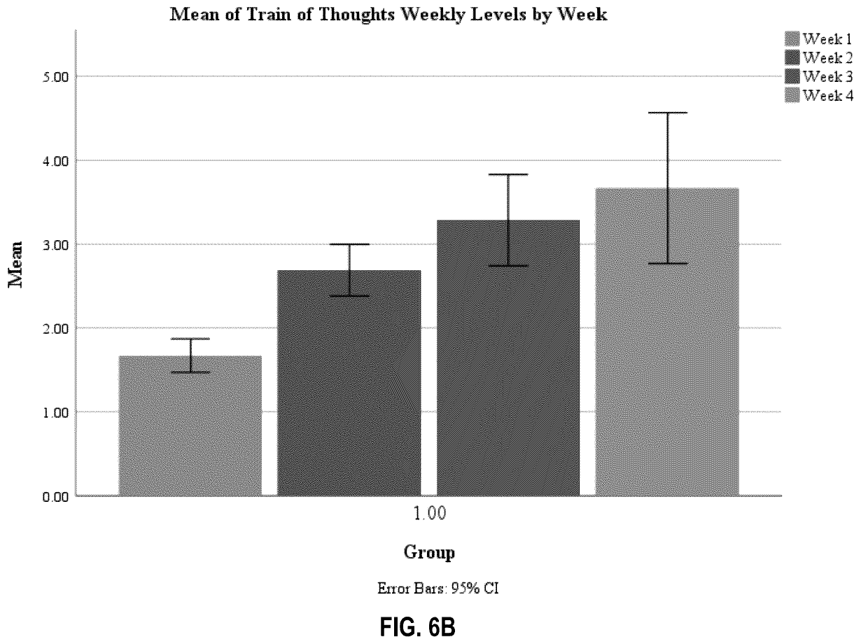

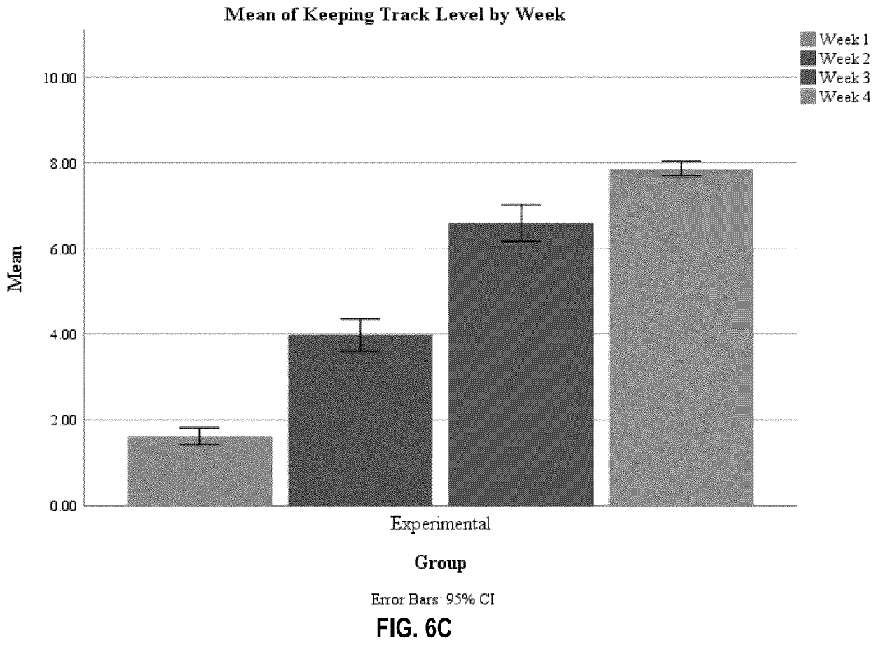

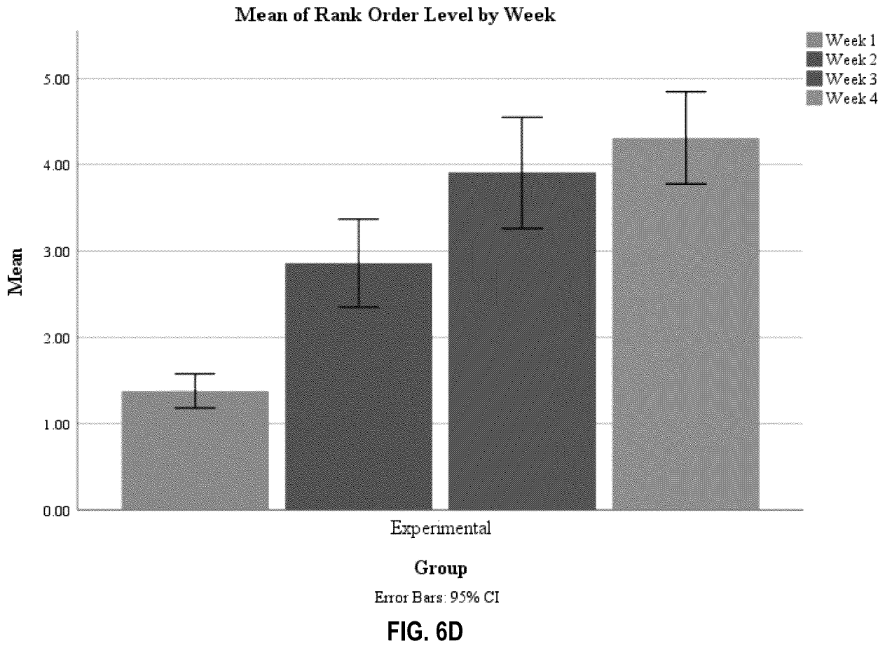

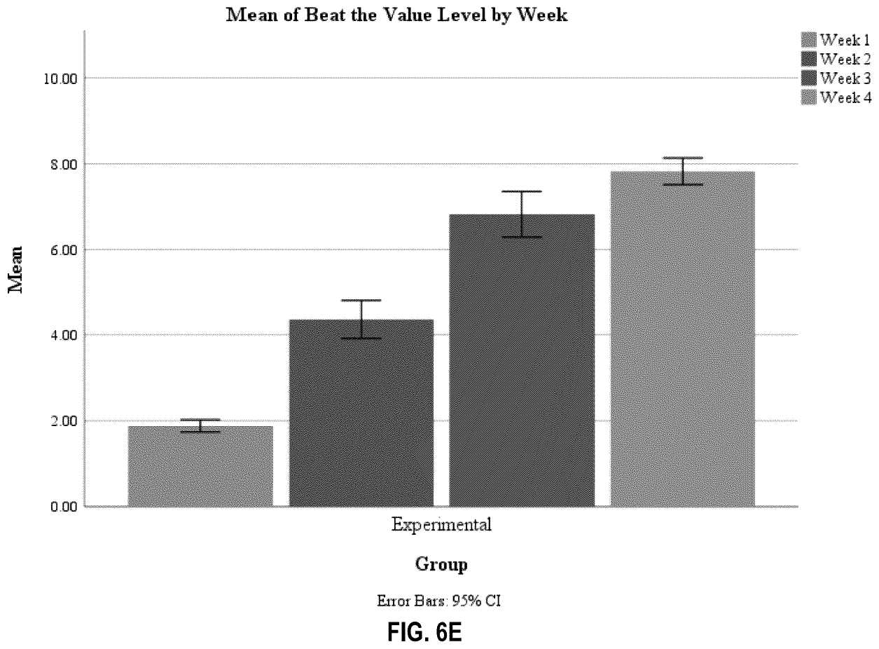

During weeks 2-4 there was an observed significant correlation between participant score and game level (see FIG. 6A and Tables 5a-c). During Week 2 of This or That the average level for experimental group participants was 4.7 and the average score was 89.86%. During this week, the observed correlation between participant score and game level was [r(14)=0.587, p=0.027]. During Week 3 of This or That the average level for experimental group participants was 7.04 and the average score was 95.50%. During this week, the observed correlation between participant score and game level was [r(14)=0.918, p<0.001]. During Week 4 of This or That the average level for experimental group participants was 7.88 and the average score was 96.71%. During this week, the observed correlation between participant score and game level was [r(14)=0.762, p=0.002].

TABLE 5a
This or That Week Two Correlation: Score × Level.
Correlations

| | | Week 2 | Week 2 | Week 2 |
|---|---|---|---|---|
| | | Score | Level | Time |
| Week 2 | Pearson Correlation | 1 | .587* | .198 |
| Score | Sig. (2 tailed) | | .027 | .497 |
| | N | 14 | 14 | 14 |
| Week 2 | Pearson Correlation | .587* | 1 | .394 |
| Level | Sig. (2-tailed) | 0.27 | | .164 |
| | N | 14 | 14 | 14 |
| Week 3 | Pearson Correlation | .198 | .394 | 1 |
| Time | Sig. (2-tailed) | .497 | .164 | |
| | N | 14 | 14 | 14 |

** Correlation is significant at the 0.01 level (2-tailed).

TABLE 5b
This or That Week Two Correlation: Score × Level.

| | | Week 3 | Week 3 | Week 3 |
|---|---|---|---|---|
| | | Score | Level | Time |
| Week 3 | Pearson Correlation | 1 | .918** | .157 |
| Score | Sig. (2 tailed) | | <.001 | .593 |
| | N | 14 | 14 | 14 |
| Week 3 | Pearson Correlation | .918** | 1 | .024 |
| Level | Sig. (2-tailed) | <.001 | | .936 |
| | N | 14 | 14 | 14 |
| Week 3 | Pearson Correlation | .157 | .024 | 1 |
| Time | Sig. (2-tailed) | .593 | .936 | |
| | N | 14 | 14 | 14 |

**Correlation is significant at the 0.01 level (2-tailed).

TABLE 5c
This or That Week Four Correlation: Score × Level.

| | | Week 4 | Week 4 | Week 4 |
|---|---|---|---|---|
| | | Score | Level | Time |
| Week 4 | Pearson Correlation | 1 | .762** | .214 |
| Score | Sig. (2 tailed) | | .002 | .463 |
| | N | 14 | 14 | 14 |
| Week 4 | Pearson Correlation | .762** | 1 | .290 |
| Level | Sig. (2-tailed) | .002 | | .315 |
| | N | 14 | 14 | 14 |
| Week 4 | Pearson Correlation | .214 | .290 | 1 |
| Time | Sig. (2-tailed) | .463 | .315 | |
| | N | 14 | 14 | 14 |

**Correlation is significant at the 0.01 level (2-tailed)

### Train of Thoughts

During weeks one there was an observed significant correlation between participant score and game level (see FIG. 6B and Table 6). During Week 1 of Train of Thoughts the average level for experimental group participants was 1.67 and the average score was 66.14%. During this week, the observed correlation between participant score and game level was [r(14)=0.545, p=0.044].

TABLE 6
Train of Thoughts Week One Correlation: Score × Level.

| | | Week 1 | Week 1 | Week 1 |
|---|---|---|---|---|
| | | Score | Level | Time |
| Week 1 | Pearson Correlation | 1 | .545* | .167 |
| Score | Sig. (2 tailed) | | .044 | .568 |
| | N | 14 | 14 | 14 |
| Week 1 | Pearson Correlation | .545* | 1 | .377 |
| Level | Sig. (2-tailed) | .044 | | .184 |
| | N | 14 | 14 | 14 |
| Week 1 | Pearson Correlation | .167 | .377 | 1 |
| Time | Sig. (2-tailed) | .568 | .184 | |
| | N | 14 | 14 | 14 |

*Correlation is significant at the 0.05 level (2-tailed).

### Keeping Track

During Week 1 of Keeping Track the average level for experimental group participants was 1.62, the average score was 86.64%, and the average time was 2 minutes and 15 seconds. During this week, the observed correlation between participant score and recorded time was [r(14)=0.637, p=0.014]. During Week 2 of Keeping Track the average level for experimental group participants was 3.97, the average score was 94.21%, and the average time was 3 minutes and 10 seconds. During this week, the observed correlation between participant recorded time and game level was [r(14)=0.593, p=0.025]. These results are depicted in Tables 7a-b and FIG. 6C.

TABLE 7a
Keeping Track Week One Correlation: Score × Time.

| | | Week 1 | Week 1 | Week 1 |
|---|---|---|---|---|
| | | Score | Level | Time |
| Week 1 | Pearson Correlation | 1 | .478 | .637* |
| Score | Sig. (2 tailed) | | .084 | .014 |
| | N | 14 | 14 | 14 |
| Week 1 | Pearson Correlation | .478 | 1 | .489 |
| Level | Sig. (2-tailed) | .084 | | .076 |
| | N | 14 | 14 | 14 |
| Week 1 | Pearson Correlation | .637* | .489 | 1 |
| Time | Sig. (2-tailed) | .014 | .076 | |
| | N | 14 | 14 | 14 |

*Correlation is significant at the 0.05 level (2-tailed).

TABLE 7b
Keeping Track Week Two Correlation: Level × Time.

| | | Week 2 | Week 2 | Week 2 |
|---|---|---|---|---|
| | | Score | Level | Time |
| Week 2 | Pearson Correlation | 1 | .367 | −.004 |
| Score | Sig. (2 tailed) | | .196 | .989 |
| | N | 14 | 14 | 14 |
| Week 2 | Pearson Correlation | .367 | 1 | .593* |
| Level | Sig. (2-tailed) | .196 | | .025 |
| | N | 14 | 14 | 14 |
| Week 2 | Pearson Correlation | −.004 | .593* | 1 |
| Time | Sig. (2-tailed) | .989 | .025 | |
| | N | 14 | 14 | 14 |

*Correlation is significant at the 0.05 level (2-tailed).

### Rank Order

During week one there was another observed significant correlation between participant score and game level (see FIG. 6D and Table 8a). During Week 1of Rank Order the average level for experimental group participants was 1.38 and the average score was 72.07%. During this week, the observed correlation between participant score and game level was [r(14)=0.683, p=0.007]. During Week 2 of Rank Order the average level for experimental group participants was 2.86, the average score was 66.14%, and the average time was 1 minute and 34 seconds. During this week, the observed correlation between participant recorded time and game level was [r(14)=0.576, p=0.031].

TABLE 8a
Rank Order Week One Correlation: Score × Level.

| | | Week 1 | Week 1 | Week 1 |
|---|---|---|---|---|
| | | Score | Level | Time |
| Week 1 | Pearson Correlation | 1 | .683** | .192 |
| Score | Sig. (2 tailed) | | .196 | .989 |
| | N | 14 | 14 | 14 |
| Week 1 | Pearson Correlation | .683** | 1 | .373 |
| Level | Sig. (2-tailed) | .007 | | .190 |
| | N | 14 | 14 | 14 |
| Week 1 | Pearson Correlation | .192 | .373 | 1 |
| Time | Sig. (2-tailed) | .510 | .190 | |
| | N | 14 | 14 | 14 |

**Correlation is significant at the 0.01 level (2-tailed).

TABLE 8b
Rank Order Week Two Correlation: Level × Time.

| | | Week 2 | Week 2 | Week 2 |
|---|---|---|---|---|
| | | Score | Level | Time |
| Week 2 | Pearson Correlation | 1 | −.029 | −.239 |
| Score | Sig. (2 tailed) | | .922 | .410 |
| | N | 14 | 14 | 14 |
| Week 2 | Pearson Correlation | −.029 | 1 | .576* |
| Level | Sig. (2-tailed) | .922 | | .031 |
| | N | 14 | 14 | 14 |
| Week 2 | Pearson Correlation | −.239 | .576* | 1 |
| Time | Sig. (2-tailed) | .410 | .031 | |
| | N | 14 | 14 | 14 |

*Correlation is significant at the 0.05 level (2-tailed).

### Beat the Value

There was no significant correlation between participants' recorded time and level during weeks 1-4 of Beat the Value. However, during week three there was an observed significant correlation between participant score and game level (see FIG. 6E and Table 9). During Week 3 of Beat the Value the average level for experimental group participants was 6.82 and the average score was 88.79%. During this week, the observed correlation between participant score and game level was [r(14)=0.750, p=0.002].

TABLE 9
Beat the Value Week Three Correlation: Score × Level.

| | | Week 3 | Week 3 | Week 3 |
|---|---|---|---|---|
| | | Score | Level | Time |
| Week 3 | Pearson Correlation | 1 | .750** | −.439 |
| Score | Sig. (2 tailed) | | .002 | .116 |
| | N | 14 | 14 | 14 |
| Week 3 | Pearson Correlation | .750** | 1 | −.347 |
| Level | Sig. (2-tailed) | .002 | | .224 |
| | N | 14 | 14 | 14 |
| Week 3 | Pearson Correlation | −.439 | −.347 | 1 |
| Time | Sig. (2-tailed) | .116 | .224 | |
| | N | 14 | 14 | 14 |

**Correlation is significant at the 0.01 level (2-tailed).

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A computer-implemented system for executive function rehabilitation, comprising:
a set of instructions executable by a processor, such that the processor is configured to:
execute an assessment session, the assessment session configured to derive an initial assessment of an individual related to execution function;
provide a plurality of task assessments related to at least one of planning, organization, or cognitive flexibility, the plurality of task assessments ordered based on results of the initial assessment;
access results from the plurality of task assessments;
generate a dashboard based on at least a portion of the results from the plurality of task assessments, the dashboard providing a plurality of games for generating game results from the individual, the plurality of games focusing on cognitive flexibility, planning, and organization; and
output via the dashboard at least one score associated with the game results and recommended resources based on the at least one score.

2. The computer-implemented system of claim 1, wherein the processor is further configured to:
generate an additional assessment session to derive a follow-up assessment of the individual related to execution function; and
compare the results of the initial assessment to the follow-up assessment to weigh improvement of the individual.
